(19) United States
(12) Patent Application Publication
(10) Pub. No.: US 2017/0039878 A1
(43) Pub. Date: Feb. 9, 2017

(54) Wellness Navigation Expert System

(71) Applicants: Paul L. Evans, Atlanta, GA (US); Aimon T. Kopera, Atlanta, GA (US)

(72) Inventors: Paul L. Evans, Atlanta, GA (US); Aimon T. Kopera, Atlanta, GA (US)

(21) Appl. No.: 14/820,944

(22) Filed: Aug. 7, 2015

(51) Int. Cl. G09B 19/00; A61B 5/16

(52) U.S. Cl. CPC G09B 19/00; G09B 19/0092; A61B 5/165

(57) ABSTRACT

A system and method are disclosed of exemplary embodiments for activating, navigating and controlling wellness protocols generated by expert systems algorithmic analysis of user qualitative and quantitative input data parameters in nine lifestyle areas: food, drink, movement, mind, wear, space, rest, self-care and social collaboration using a graphical systems interface and a user input device (mechanical pointing or touch) for displaying and navigating lifestyle/wellness protocols and purchasing related products and services and conducting social collaboration. The system operates on smart phones, tablet computers and desktop computers where lifestyle/wellness protocols are generated based on expert systems algorithmic analysis of qualitative and quantitative input data parameters (lifestyle data and bio-medical data). And, a method is disclosed for receiving qualitative and quantitative input including, but not limited to, wellness and health history questionnaires and bio-medical data and protocols and accessing and integrating wellness data and protocol content and social interactions within the system.

### CROSS-REFERENCE TO RELATED APPLICATIONS

This application claims the benefit of U.S. Provisional Application Ser. No. 62/042,578, filed Aug. 27, 2014, under 35 U.S.C. §119(e) the contents are incorporated herein by reference.

### BACKGROUND OF THE INVENTION

The links among healthy populations, productive workforces and economic growth are well established. According to the world economic forum, worldwide, $47 trillion of cumulative output loss can be expected in the next two decades due to employee chronic disease. Currently, 36 million deaths annually or more than 60% of global deaths are due to cardiovascular diseases, diabetes, cancer and chronic respiratory diseases. However, it is also known that tracking and monitoring key risk factors may prevent 80% of cardiovascular problems and 40% of cancers.

The research is clear. The relationship among lifestyle choices such as the food we eat, what we drink, what we breathe, how we move, our mental, social and physical living activities and environments, all influence gene expression and the development of chronic illness. The relationship is both direct and causal. For a better, longer life optimal lifestyle choices are required.

There is therefore a great need for a method and system that can identify key lifestyle risk factors in individuals and provide individualized plans protocols to improve the management of chronic disease and facilitate the maintenance of personal wellness and prevention.

The field of the present invention relates generally to “expert systems” methodologies and systems used to promote healthy lifestyles and wellness. Expert systems were one of the first areas to be commercially fruitful within the field known as artificial intelligence. These systems are now operational in many areas, including medicine and healthcare. Many expert systems are rule-based and are relatively simple, easy-to-understand, and efficient in specific diagnostic situations. These systems rely on two components: a knowledge base (content, for example, lifestyle/wellness protocols for healthy living) and “inference engine” algorithms to analyze user input and other data to generate or determine the appropriate protocols to recommend.

For the most part, conventional expert systems rely on structured data. Structured data refers to information with a high degree of organization, such that inclusion in a relational database is seamless and readily searchable by simple, straightforward search engine algorithms or other search operations. Data in spreadsheets, for example, can be considered structured data, which can be quickly scanned and categorized in a relational database system.

Expert systems are also emerging that can use unstructured data. Narrative language in the form of books, medical journals or even everyday conversation is primarily unstructured data. The problem that unstructured data presents is one of volume. Because the pool of information is so large, current data mining techniques and web search engines use techniques like metadata with harvesting technology that employs multiple threads to mass-harvest scalable quantities of unstructured data. Harvests are based on multiple system-developed queries with results (web pages, PDF″s, XLS, PPT, XML, etc.) qualified through customizable filters. Scoring algorithms index the information based on relevancy to further qualify the documents returned systems, ensuring the system is seeing only super-relevant content. The final system interface displays the qualified results in a searchable database based on customizable facets (URL, file type, source category, people mentioned, places mentioned, companies mentioned, custom keywords, etc.).

Through analysis of both structured and unstructured data some expert systems allow for machine learning where knowledge accumulates in the system from increased user enrollment and activities and the system updates the existing knowledge (for example, relative to the current disclosure, a protocol database) based on increased user data, interaction and outcomes.

Today a wide range of commercially available software can be used to construct medical expert systems. These tools can be used to build medical protocols based on identifying patient problems and indicators (e.g., from various questionnaires, health risk assessments, laboratory tests and even gene sequencing) to construct a set of inference rules; identify interventions to address the patient problems; build rules for the interventions; and, describe a set of desired outcomes. In addition to off-the-shelf software, code can be written, debugged, and built into a new applications for expert system protocol engines.

A variety of lifestyle/wellness software programs and services are also available that implement expert-like systems and offer lifestyle/wellness plans where data from activity trackers and other information is collected from, or entered by, the user on computers and analyzed by algorithms to generate lifestyle/wellness plans. These plans are often focused on areas such as weight loss and peak performance. And, they may include protocols or recommendations for activity tracking and diet monitoring among other wellness activities.

For example, in related art to the current disclosure, the following methods and systems are generally directed toward tracking and monitoring quantitative indices:

U.S. Pat. No. 2015/0262497 to Landau, et al. discloses methods and systems for customizing wellness plans to systems based on activity detected by activity tracking devices. Landau discloses the use of quantitative system input wherein the data provided are dependent on “personal input parameters” that establish the quantitative baseline.

U.S. Pat. No. 2014/0342328 to Pacione, et al. discloses a nutrition and activity management system that monitors energy expenditure through the use of a body mounting sensing apparatus as a means for calorie control, weight control or general fitness. In this disclosure the system analyzes baseline data and quantitative system goals to generate suggested activity and suggested behavior modification to improve progress toward goals.

U.S. Pat. No. 2012/0094258 to Langheier, et al. discloses a system and method for automated eating and activity planning providing personalized wellness recommendations and facilitating ordering of healthy meals.

These applications are important but necessarily limited because preventive medicine and wellness protocol-based intervention is a complex medical problem that entails any number of wellness domain tasks involving food, drink, movement, mind, rest, self-care and social collaboration. Each of these tasks may be addressed by one or more self-contained applications such as the activity tracker applications mentioned above for the “move” or movement domain. While a tremendous amount of data is generated by these single activity devices and applications, it is not always readily clear how to integrate them and apply them in an intuitive way to understand and assess comprehensive everyday activity and health habits across food, drink, move, mind, wear, space, rest, self-care and social collaboration wellness domains.

In effect, automation of only a single task such as activity tracking, nutrition tracking, and even ordering of healthy meals does not take into consideration the method and system integration of the multiplicity of possible quantitative and qualitative inputs (e.g., from various questionnaires, health risk assessments, laboratory tests and even gene sequencing) along with the variety of outcome wellness/lifestyle domain protocols (e.g., food, drink, movement, mind, wear, space, rest, self-care, and social collaboration) that inform comprehensive approaches to lifestyle modification and wellness.

Further, at any given moment, individuals are potentially eligible for new protocols or guidelines, based on continued interaction and the variety of wellness domains. The multiplicity of possible domain tasks suggest that users need a flexible and robust method and system for implementing multiple applications for automation of multiple domain tasks in food, drink, movement, mind, wear, space, rest, self-care and social collaboration domains quickly and easily. And, to be effective systems should be able to navigate and control comprehensive domain knowledge for all the required domain tasks of food, drink, movement, mind, space, rest, self-care and social collaboration and not just focus on one single domain task and to be able to update all domain tasks in relation to progress in independent domains.

Today, a vast range of customized wellness plans are available in expert systems computing environments that focus a limited number of domain tasks. However, the lack of systems integration capability and inclusion of numerous domain tasks involving food, drink, movement, mind, wear, rest, self-care and social collaboration necessarily compromises their recommended outcomes. Counting calories and counting footsteps are quantitative indices that need to be considered along with many other qualitative and quantitative parameters that exist in other wellness domain tasks.

Some wellness/lifestyle applications come ready with their own system interfaces for desktop, tablet computers and smart phones. This further complicates the user experience by creating multiple system interface experiences for each different application and requiring alternative system interface for protocol creation, access, control and communication. Thus there is a need for a standard method for system integration for navigating and control of lifestyle/wellness protocols with which users can immediately identify.

Given that such standard system integration would be designed for users and applications of all types, it is desirable to provide a standard system interface device that is simple, approachable and forgiving for users at all levels. Moreover, such a standard would enable the user to navigate and control collections of input data, protocols and execute access with ease from various applications.

What is needed is a way to optimize system navigation and control of lifestyle/wellness protocols from a variety of data sources, devices and systems, qualitative and quantitative. In addition, there is a need for the user to move freely and navigate among protocols for food, drink, movement, mind, wear, space, rest, and self-care and connect with other users in social collaboration.

Users want to avoid the high cost and complexity of purchasing and using disparate systems in multiple domains for lifestyle/wellness protocols. Therefore, lifestyle/wellness applications should have as much wellness domain knowledge as possible across all the lifestyle/wellness domain tasks. Furthermore, users want to be able to migrate protocols from disparate applications to more integrated systems.

No effective methods have been developed focused on system integration and navigation among the various lifestyle/wellness protocols in systems for food, drink, movement, mind, wear, space, rest, self-care, and social collaboration from a “systems integration” perspective. It is desirable to provide such a method and system for lifestyle/wellness navigation for individualized use and social collaboration that overcome the limitations of the existing systems and methods and it is to this end that the disclosure is directed.

### BRIEF SUMMARY OF THE INVENTION

The present invention relates generally to digital wellness expert system methodologies and systems used to promote healthy lifestyles. The present invention is novel and instructs a method and system interface for generating, activating and controlling wellness protocols in nine lifestyle areas: food, drink, movement, mind, wear, space, rest, self-care and social collaboration and for integration, activation and control of disparate wellness expert systems wellness protocols generated by expert systems algorithmic analysis of user input data parameters.

An integrated lifestyle management method and system is disclosed. Through a comprehensive assessment of nine factors (food & supplements, drink, move, mind, wear, space, rest, self-care, and social) overall health and well-being is analyzed. Analysis of these lifestyle components identify areas of strength as well as those that need improvement. Results are used to help build effective strategies, programs and services for healthy lifestyles. Participants achieve optimal wellness by utilizing comprehensive lifestyle assessments, protocols for healthy living, web-based wellness resources and targeted lifestyle programs and services.

Powerful analytics provide insights and access to lifestyle data, programs and services anytime, anywhere via the systems interface. Protocols include: 1) Foods, Super-foods and Supplements; 2) Drink and Hydration; 3) Mindful Movement; 4) Meditation and Contemplative Practice; 5) Sensors, Monitors and Trackers; 6) Space and Environmental Toxins; 7) Rest; 8) Self-care and detoxification; and, 10) Social Collaboration.

In various embodiments, a flexible, scalable method and system interface for interacting with a variety of systems ranging from a smart phone, to a desktop computer or a tablet PC is disclosed. A method and system interface is disclosed for the systems integration of: 1) input of wellness/lifestyle questionnaires, biological test data and genetic data to construct wellness protocols; 2) purchasing wellness/lifestyle products, services and data; 3) communication and monitoring data and information regarding wellness/lifestyle protocol content areas among system users; and 4) incorporation of external data sources and protocols from disparate expert system applications and databases.

### BRIEF DESCRIPTION OF THE DRAWINGS

Embodiments of the invention are illustrated in the drawings, in which:

FIG. 1. Illustrates the protocol generator. The system compares current lifestyle data and information with the database of optimal wellness lifestyles and identifies dissimilarities. Individualized wellness protocols are created by the system as a result of this process. Protocols can be accessed by computers, tablets or by smart phones as shown.

FIG. 2. illustrates the system databases including: 1) A wellness protocol generation engine; 2) Directories of products and services for users to purchase; 3) Protocol instructional content in each of the nine lifestyle areas of food, drink, movement, mind, wear, space, rest, self-care and social collaboration; and, 4) A communication database module for system collaboration. Databases can be accessed by computers, tablets or by smart phones as shown.

FIG. 3. Illustrates the system interface for generation, activation, navigational control of lifestyle/wellness protocols and API and Internet content integration of lifestyle/wellness data and protocols generated by disparate expert systems;

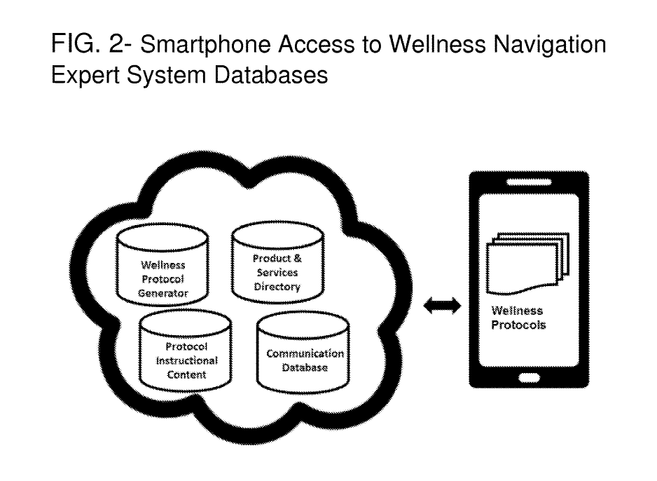

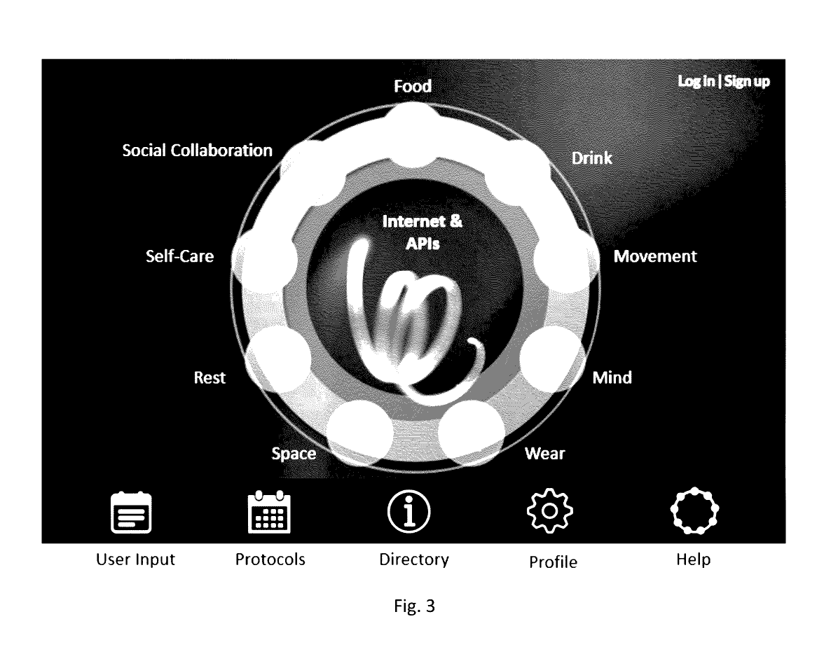

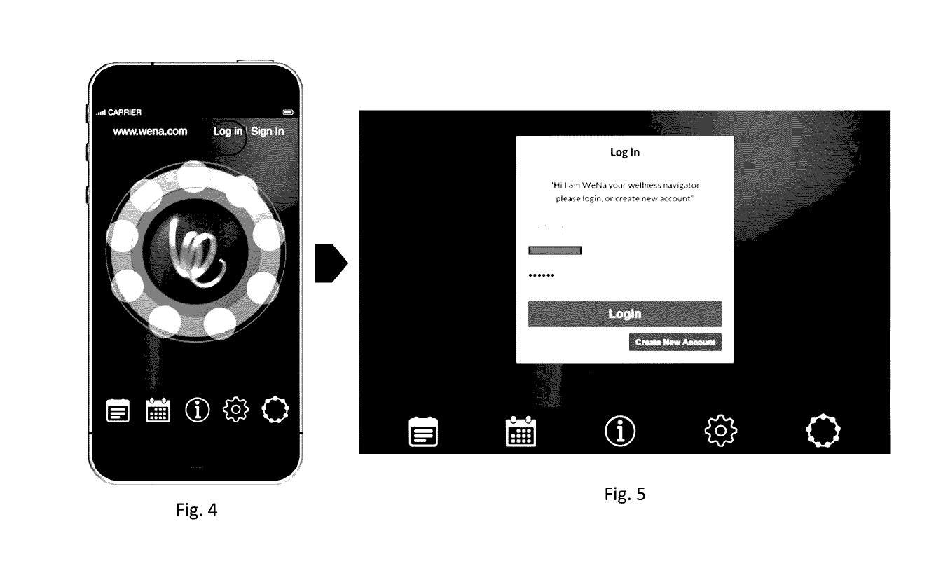

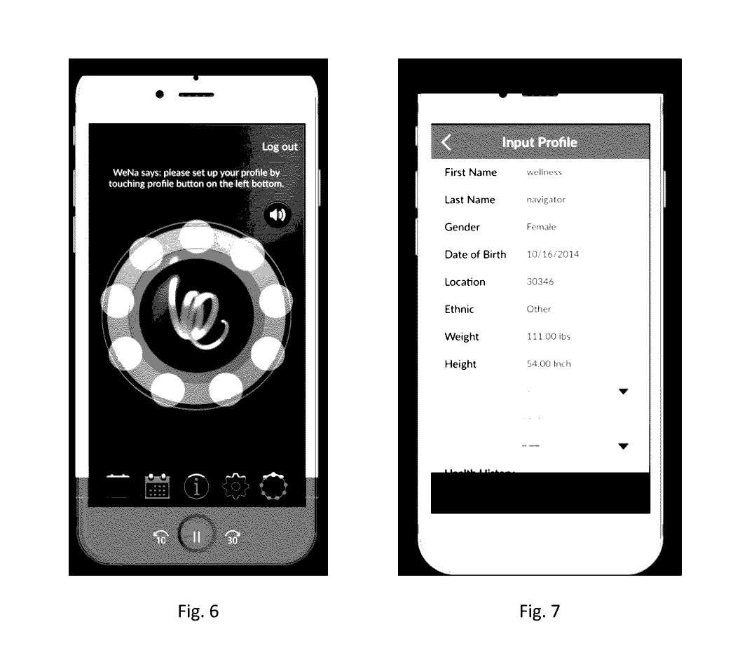

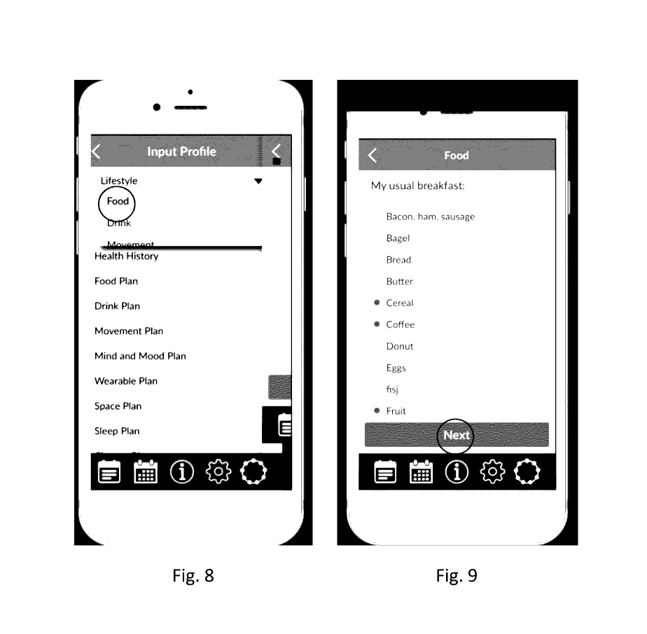

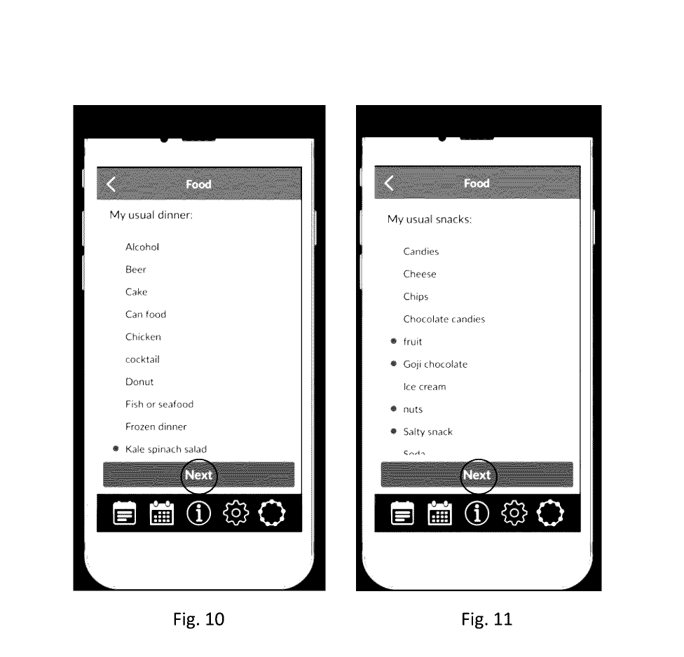

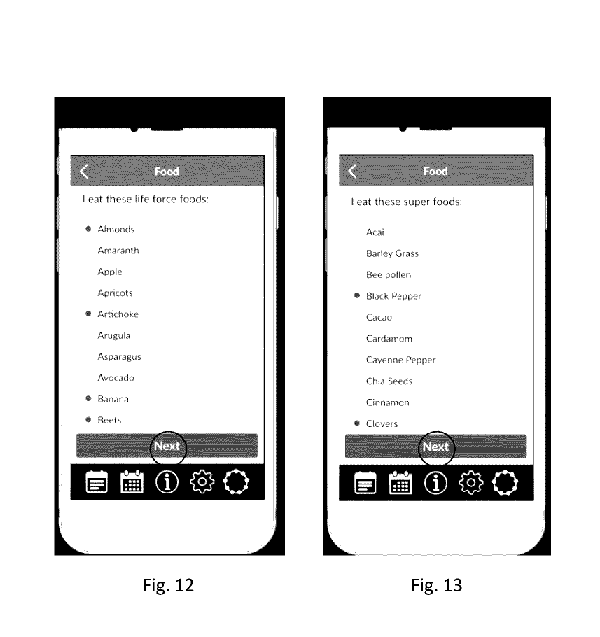

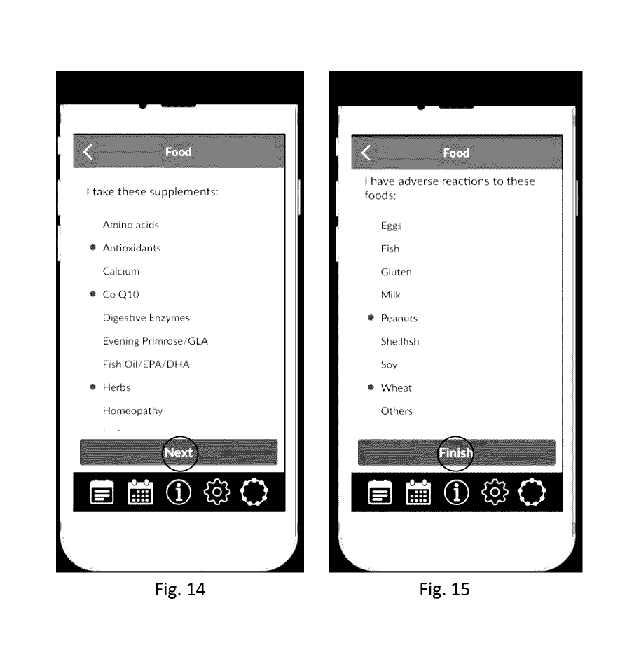

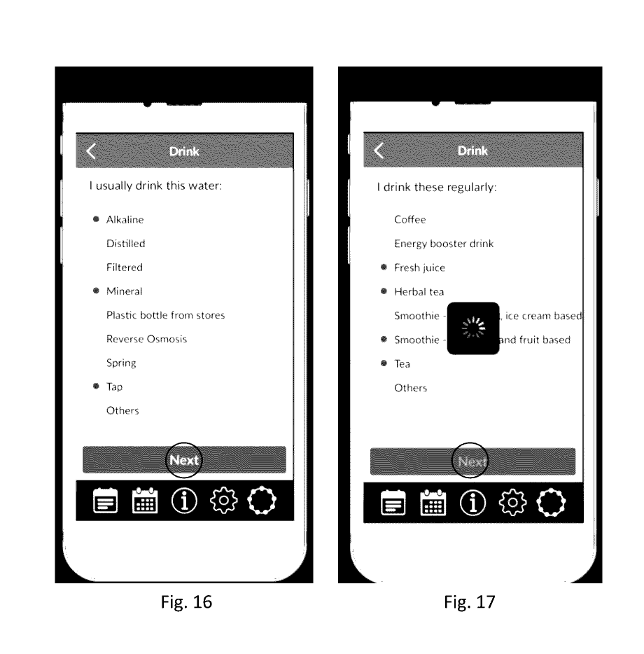

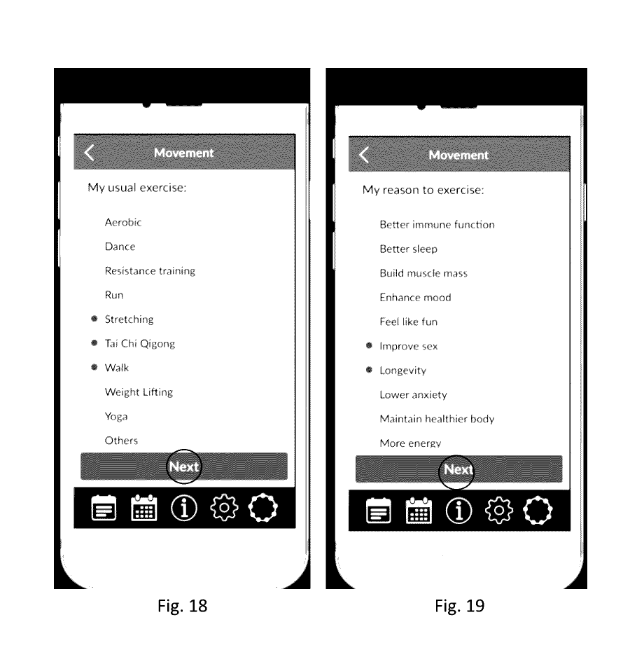

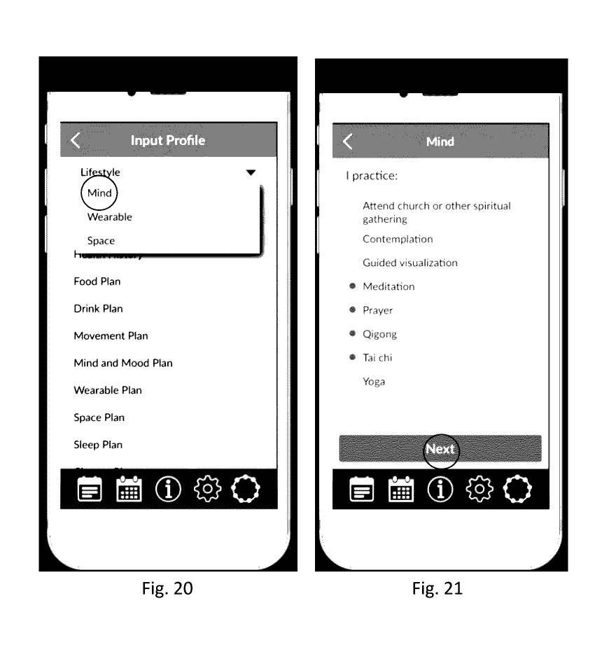

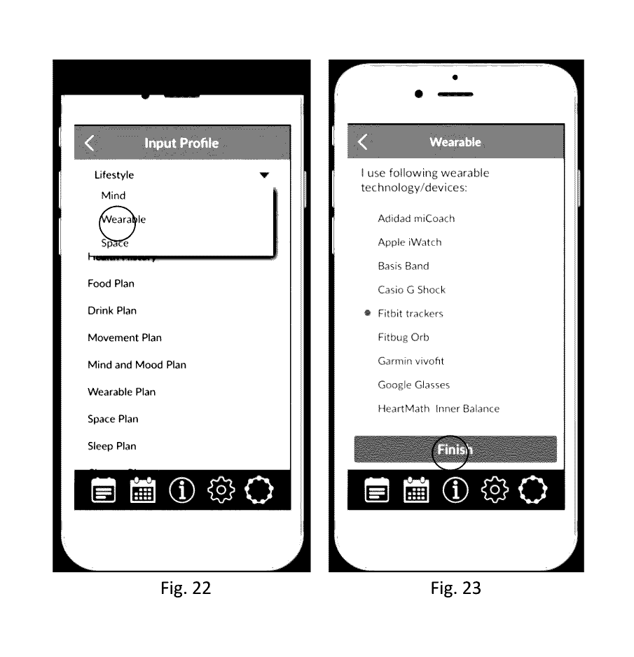

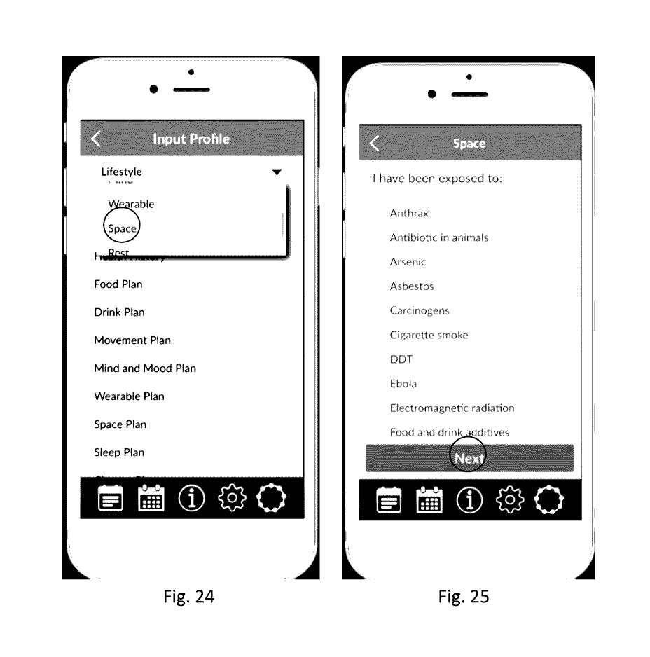

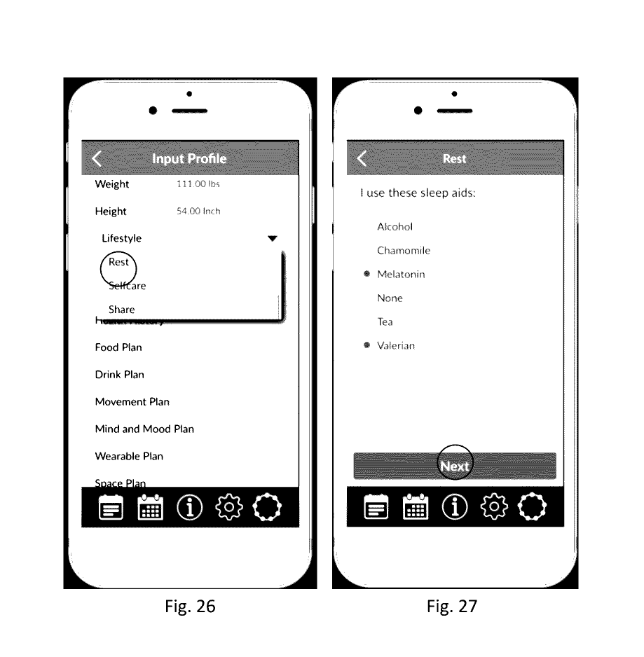

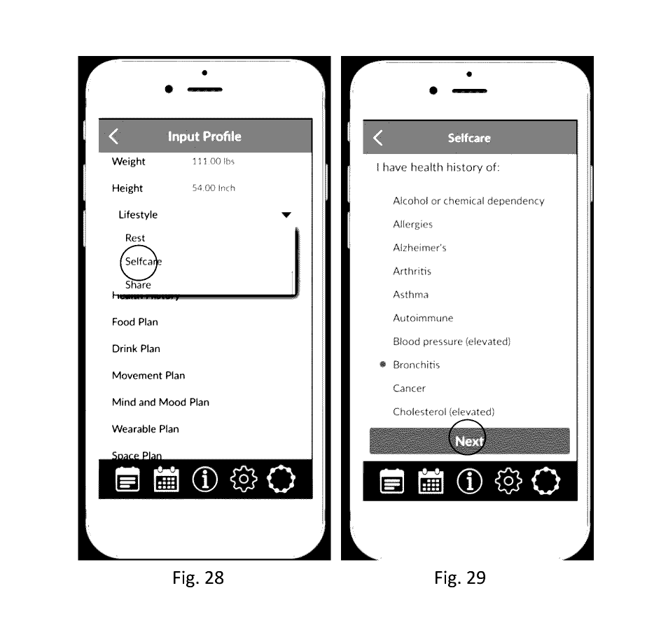

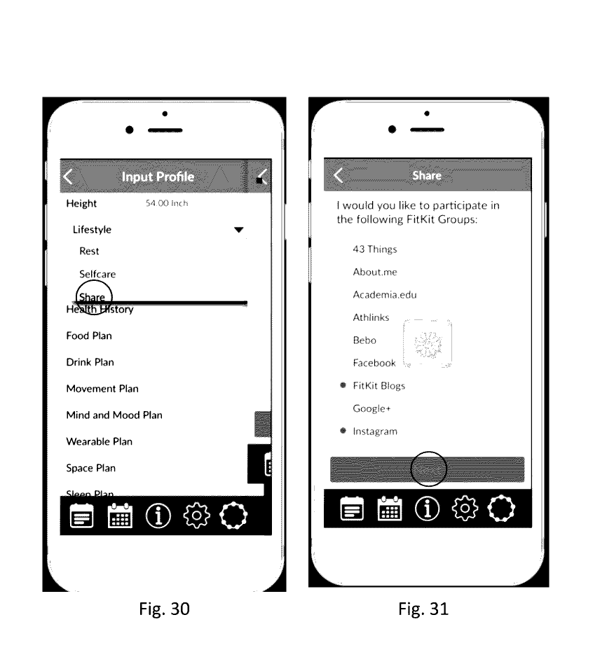

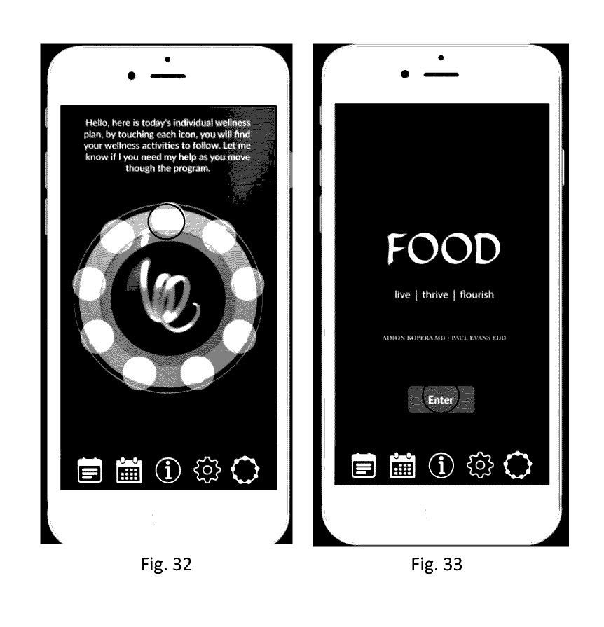

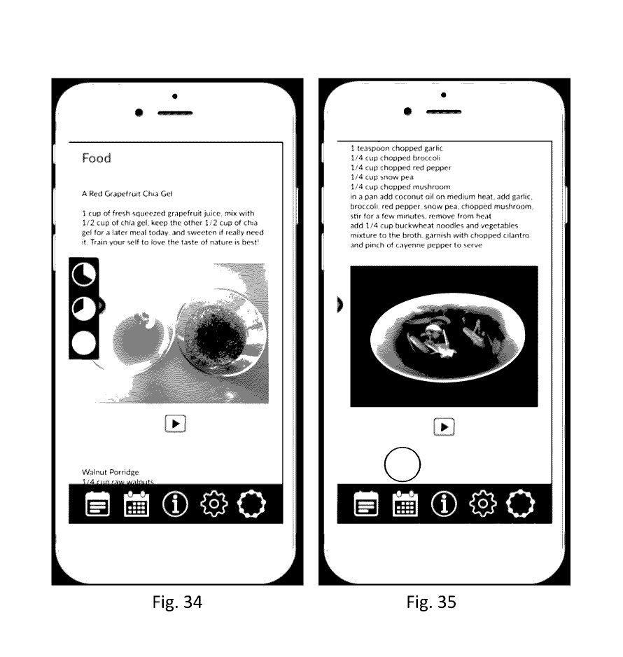

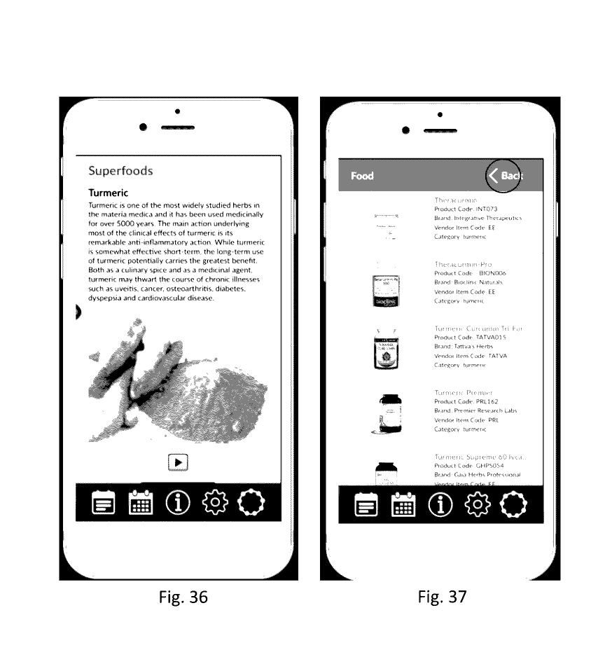

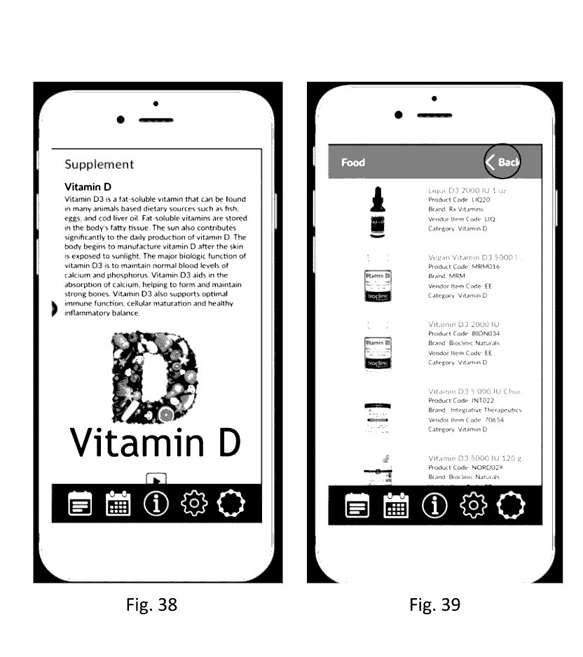

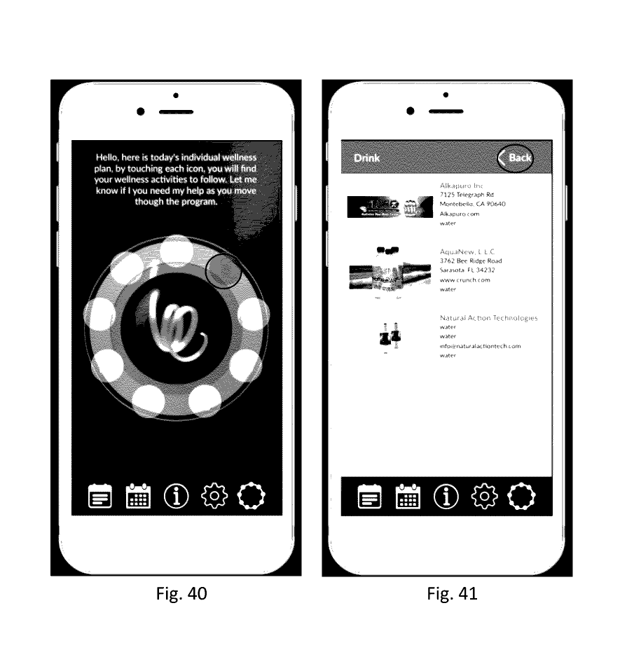

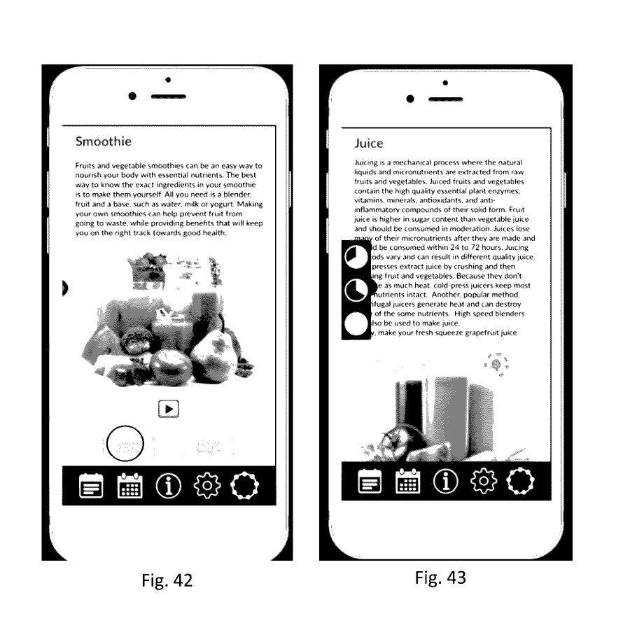

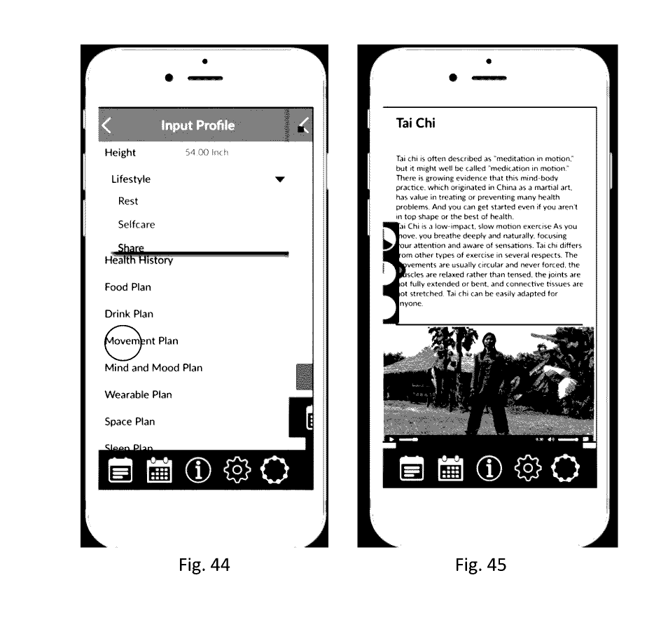

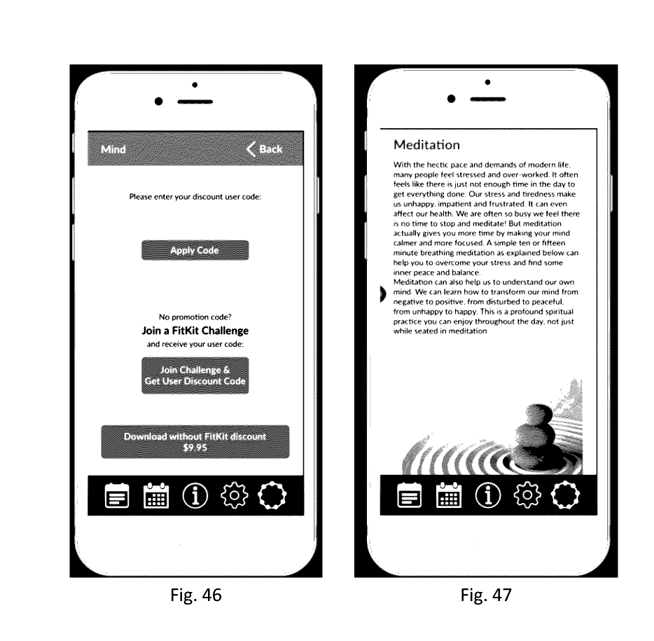

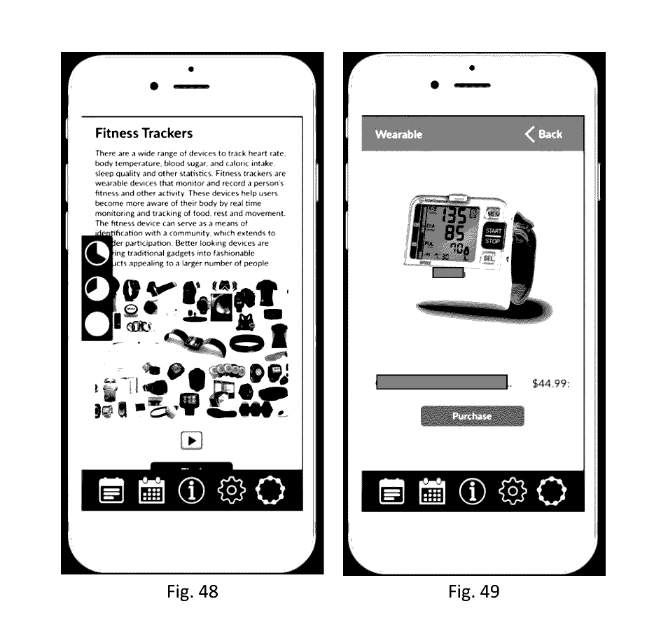

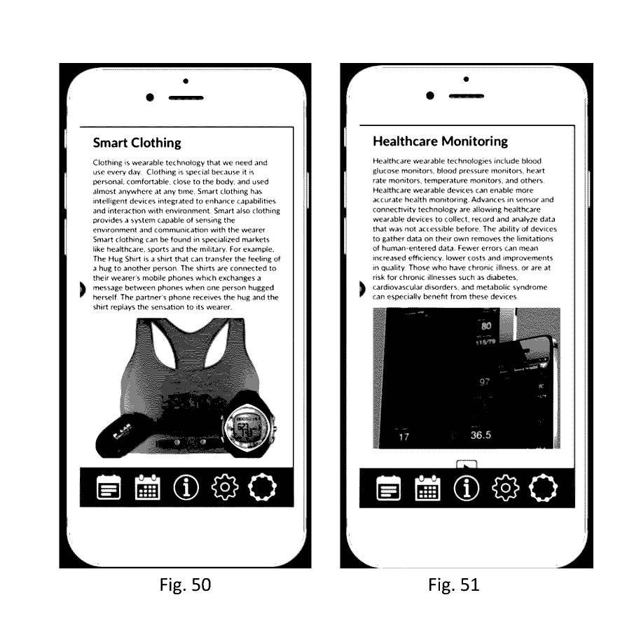

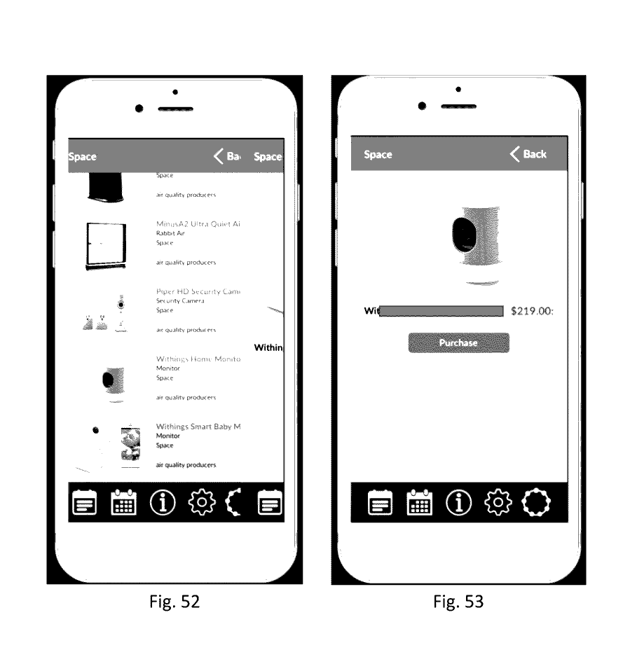

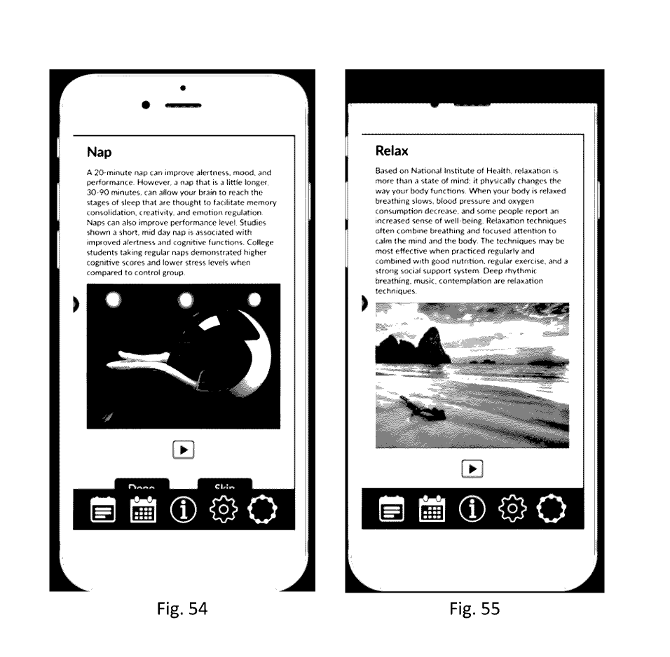

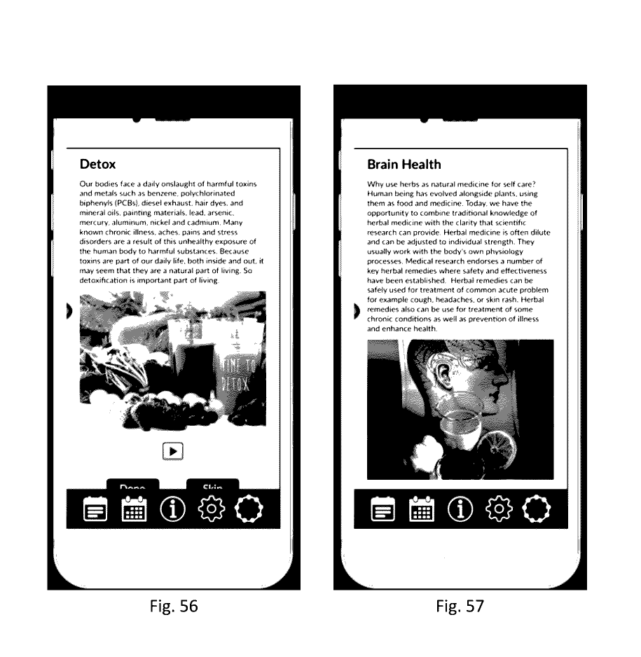

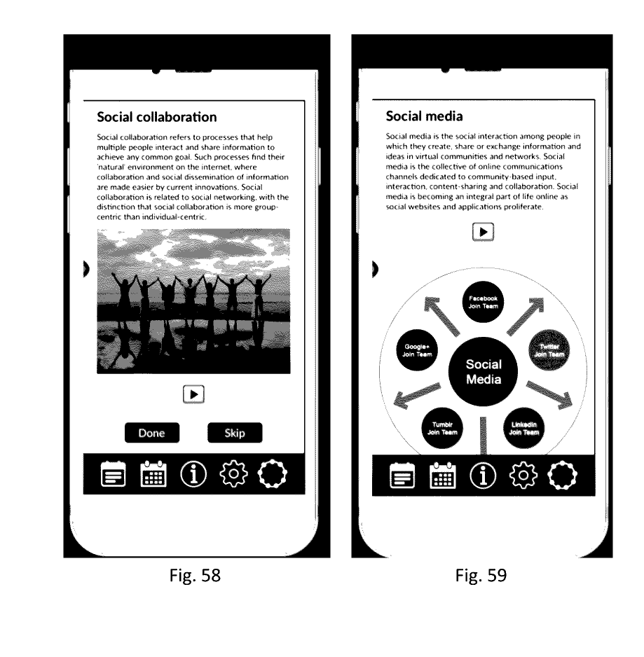

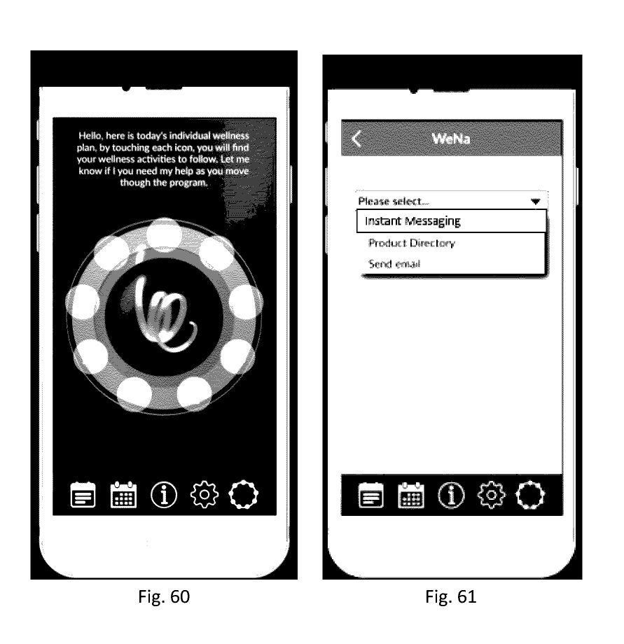

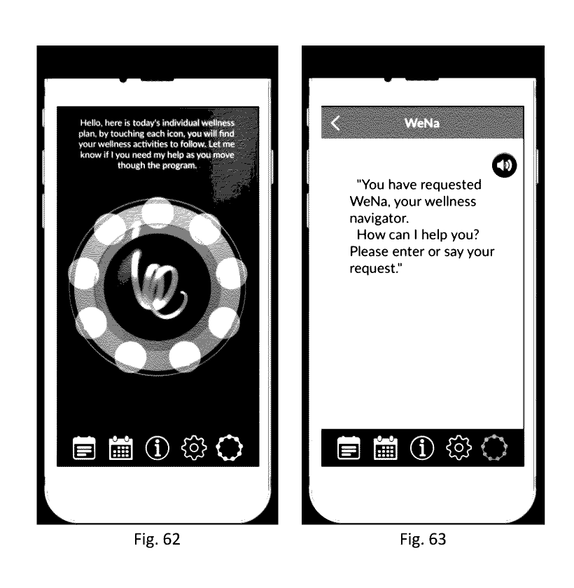

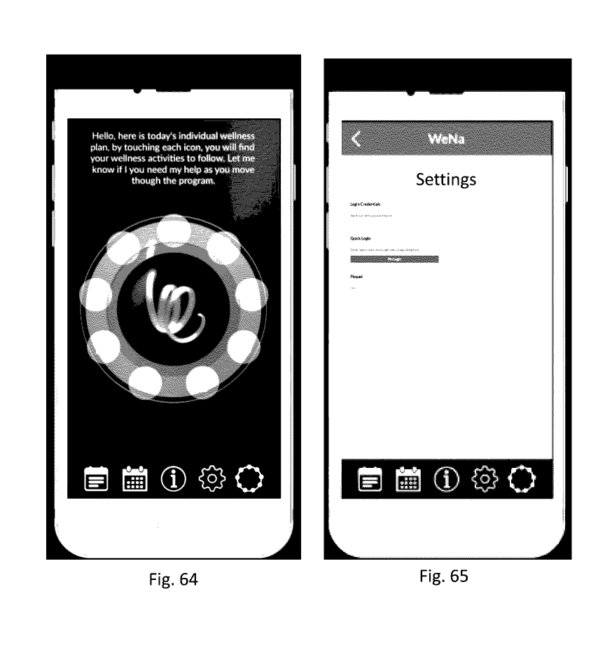

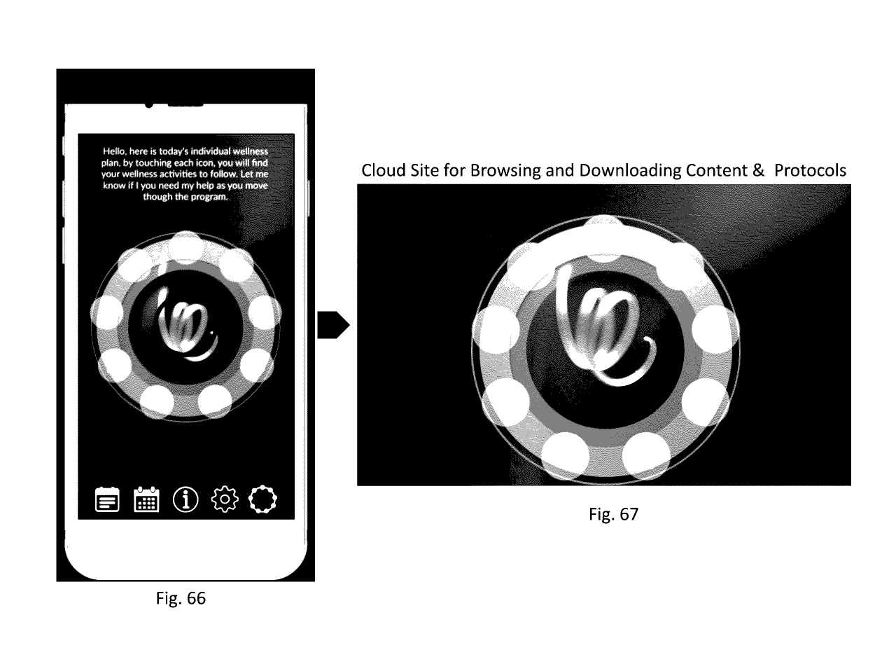

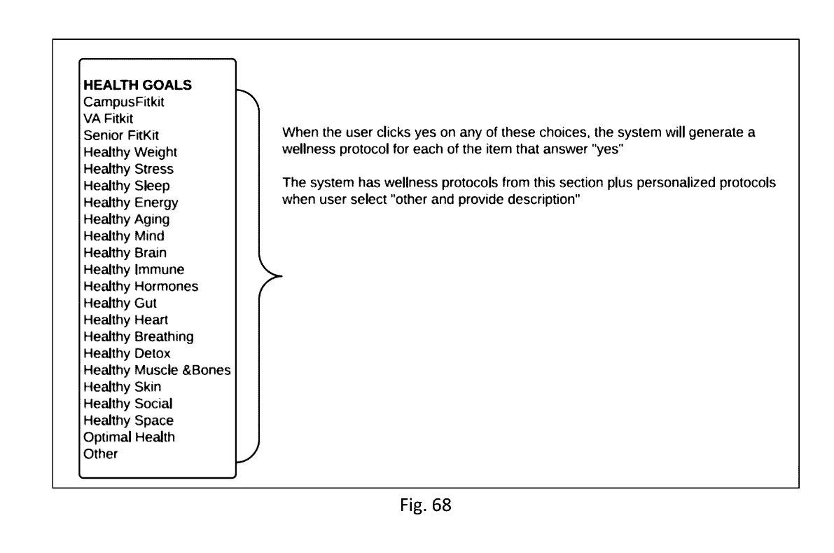

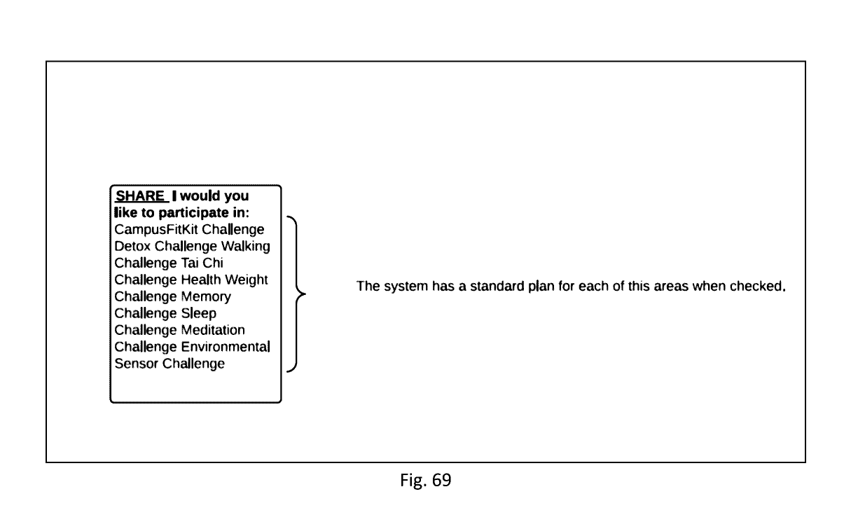

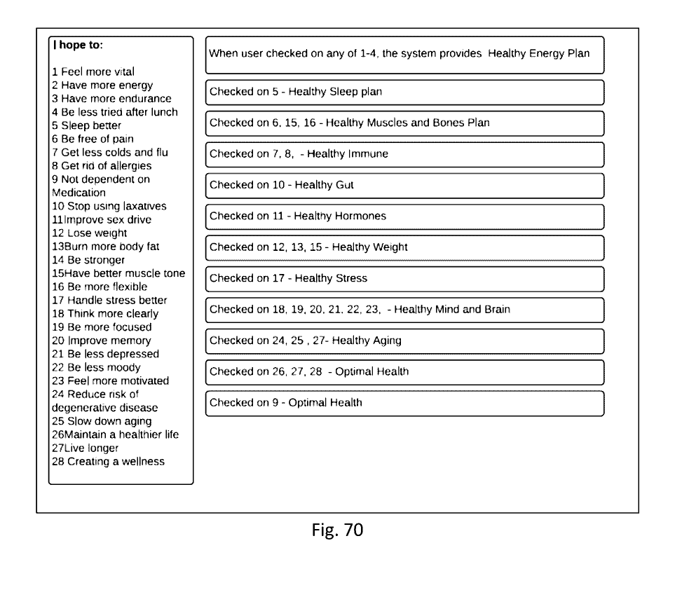

FIGS. 2-3 illustrate an example the system interface and initial profile input procedure for a system for providing navigation of lifestyle/wellness protocols generated by expert systems;

FIGS. 4-5 illustrate the login procedure for a system for providing navigation of lifestyle/wellness protocols generated by expert systems;

FIGS. 6-7 illustrate an example the system interface food profile input procedure for a system for providing navigation of lifestyle/wellness protocols generated by expert systems;

FIGS. 8-9 further illustrate an example the system interface food profile input procedure for a system for providing navigation of lifestyle/wellness protocols generated by expert systems;

FIGS. 10-13 further illustrate an example the system interface food profile input procedure for a system for providing navigation of lifestyle/wellness protocols generated by expert systems;

FIGS. 14-15 illustrates an example of the system interface for food supplements and adverse reaction input procedure for a system for providing navigation of lifestyle/wellness protocols generated by expert systems;

FIGS. 16-17 illustrates an example the system interface for drink input procedure for a system for providing navigation of lifestyle/wellness protocols generated by expert systems;

FIGS. 18-19 illustrates an example the system interface for movement input procedure for a system for providing navigation of lifestyle/wellness protocols generated by expert systems;

FIGS. 20-21 illustrates an example the system interface for mind input procedure for a system for providing navigation of lifestyle/wellness protocols generated by expert systems;

FIGS. 22-23 illustrates an example the system interface for wear input procedure for a system for providing navigation of lifestyle/wellness protocols generated by expert systems;

FIGS. 24-25 illustrates an example the system interface for space input procedure for a system for providing navigation of lifestyle/wellness protocols generated by expert systems;

FIGS. 26-27 illustrates an example the system interface for rest input procedure for a system for providing navigation of lifestyle/wellness protocols generated by expert systems;

FIGS. 28-29 illustrates an example the system interface for self-care input procedure for a system for providing navigation of lifestyle/wellness protocols generated by expert systems;

FIGS. 30-31 illustrates an example the system interface for social-collaboration input procedure for a system for providing navigation of lifestyle/wellness protocols generated by expert systems;

FIGS. 32-33 illustrates an example the system interface for food protocol for a system for providing navigation of lifestyle/wellness protocols generated by expert systems;

FIGS. 34-35 further illustrates an example the system interface for food protocol for a system for providing navigation of lifestyle/wellness protocols generated by expert systems;

FIGS. 36-37 further illustrates an example the system interface for food protocol and food supplement ordering for a system for providing navigation of lifestyle/wellness protocols generated by expert systems;

FIGS. 38-39 further illustrates an example the system interface for food protocol and food supplement ordering for a system for providing navigation of lifestyle/wellness protocols generated by expert systems;

FIGS. 40-43 illustrates an example the system interface for drink protocol and drink preparation for a system for providing navigation of lifestyle/wellness protocols generated by expert systems;

FIGS. 44-45 illustrates an example the system interface for movement protocol and movement instruction for a system for providing navigation of lifestyle/wellness protocols generated by expert systems;

FIGS. 46-47 illustrates an example the system interface for mind protocol and protocol purchasing interface for applying purchase discounts for a system for providing navigation of lifestyle/wellness protocols generated by expert systems;

FIGS. 48-49 illustrates an example the system interface for wear protocol and protocol purchasing interface for a system for providing navigation of lifestyle/wellness protocols generated by expert systems;

FIGS. 50-51 further illustrates an example the system interface for wear protocol and protocol purchasing interface for a system for providing navigation of lifestyle/wellness protocols generated by expert systems;

FIGS. 52-53 illustrates an example the system interface for space protocol and protocol purchasing interface for a system for providing navigation of lifestyle/wellness protocols generated by expert systems;

FIGS. 54-55 illustrates an example the system interface for rest protocol for a system for providing navigation of lifestyle/wellness protocols generated by expert systems;

FIGS. 56-57 illustrates an example the system interface for self-care protocol for a system for providing navigation of lifestyle/wellness protocols generated by expert systems;

FIGS. 58-59 illustrates an example the system interface for social collaboration protocol for a system for providing navigation of lifestyle/wellness protocols generated by expert systems;

FIGS. 60-61 further illustrates an example the system interface for directory and products for a system for providing navigation of lifestyle/wellness protocols generated by expert systems;

FIGS. 62-63 illustrates an example the system interface for intelligent agent protocol for a system for providing navigation of lifestyle/wellness protocols generated by expert systems;

FIGS. 64-65 illustrates an example the system interface for profile settings for a system for providing navigation of lifestyle/wellness protocols generated by expert systems;

FIGS. 66-67 illustrate an example the system interface for browsing and downloading external content for a system for providing navigation of lifestyle/wellness protocols generated by expert systems;

FIGS. 68-69 illustrate an example of a procedure for protocol creation for a system for providing navigation of lifestyle/wellness protocols generated by expert systems.

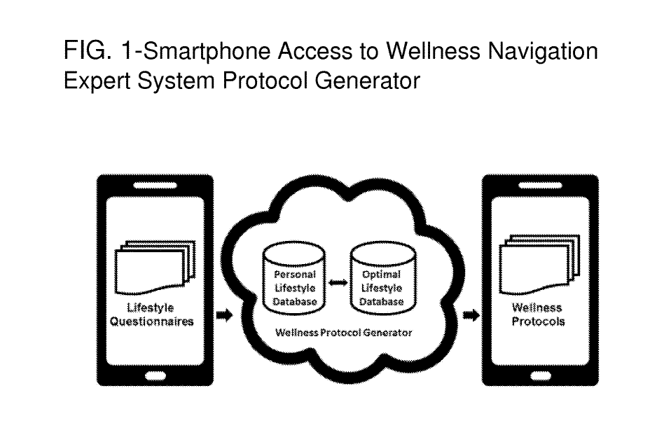

FIGS. 1-70 Illustrate exemplary embodiments of a method and system for activating wellness protocols generated by expert systems algorithmic analysis of system input data parameters in nine lifestyle areas: food, drink, movement, mind, wear, space, rest, self-care and social collaboration using a graphical system interface and a system input device (mechanical pointing or touch) displaying and navigating lifestyle/wellness protocols and ordering related products, services and conducting social collaboration.

### DETAILED DESCRIPTION OF THE INVENTION

This disclosure instructs the utility of a systems interface for generation, activation and navigational control of wellness domain protocols in nine lifestyle areas: food, drink, movement, mind, wear, space, rest, self-care and social collaboration and incorporates wellness protocols and domain knowledge from disparate applications through application program interfaces (APIs) in a manner that facilitates use of those protocols and domain knowledge on one system. In addition, access to the Internet and external databases is enabled to search, download and store relevant content in each lifestyle/wellness protocol area to augment the protocols generated, incorporated, activated and controlled by the disclosed system interface.

In the present disclosure, to navigate lifestyle/wellness protocols a computer, tablet, or smart phone is equipped with method and system interface as shown in FIG. 1. Through interacting with the icons for: food, drink, movement, mind, wear, space, rest, self-care, and social collaboration, personalized lifestyle/wellness protocols are engaged and opportunities for social collaboration are facilitated. Exemplary embodiments for wellness domain tasks of food, drink, movement, mind, wear, space, rest, self-care protocols described herein include protocol databases for text documents, graphics, video and audio information that may be inherent to the system or augmented by external programs and sources connected by Application Program Interfaces or downloaded from the internet or other databases, and browsed, searched, incorporated, and activated in the relevant protocol area.

The disclosed method and system allows the user to establish and maintain an online connection to databases for navigation or off line access to perform a lifestyle/wellness domain task and retrieve information and content, regarding individual lifestyle/wellness protocols as well as interact with other users online. The system interface is designed to support all the features and functionality needed to access, review, modify, and use lifestyle/wellness protocols in the wellness domain areas of: food, drink, movement, mind, wear, space, rest, self-care, and social collaboration. The interaction with these lifestyle/wellness protocols is facilitated by the disclosed system interface (by touch or screen pointer) with the associated icons.

The present invention relates to embodiments for a method and system of generating, activating and providing navigational access and control to one or more functions of lifestyle/wellness protocols and services in a computing systems environment.

A flexible, easy-to-use method and system for generating, activating, navigating and interacting with a variety of lifestyle/wellness protocols is disclosed. A method and system interface for generating, activating and navigational control of protocols is disclosed comprising a series of icons each mapping to one or more independent protocol databases for food, drink, movement, mind, wear, space, rest, self-care, and social collaboration.

Additional features of the present invention are described below.

A method and system is disclosed for activating, and navigating wellness protocols generated by expert systems through algorithmic analysis of system input data parameters in nine lifestyle areas: food, drink, movement, mind, wear, space, rest, self-care and social collaboration using a system input device (mechanical pointing or touch, systems interface) displaying and navigating lifestyle/wellness protocols and ordering related products, services and conducting social collaboration and activating, navigating and interacting with wellness/lifestyle protocols from disparate systems.

The disclosure instructs a method and system implemented on computer, tablets or smart phones where protocols are generated by the system based on expert systems algorithmic analysis of system input data parameters (lifestyle data and bio-medical data). Additionally, protocols in one or more lifestyle/wellness domains may be incorporated in domain areas from external systems through the systems interface.

Exemplary embodiments are disclosed for the navigation and use of lifestyle/wellness protocols generated by systems comprising of: a wellness protocol generation engine (relational database) wherein the input of wellness information identifying a user and a description of a user's lifestyle and biometrics is mapped against optimal lifestyle wellness databases in the system to determine the user's wellness needs in one or more of the lifestyle areas of food, drink, movement, mind, wear, space, rest, self-care and social collaboration; and against directories of products and services for systems to purchase lifestyle and wellness products and service.

Data file directories regarding user interest in wellness products and services are stored along with data on products and services ordered and used in the lifestyle areas of food and nutrition, drink, movement, mind, wear, space, rest, self-care and social collaboration.

Protocol instructional content in each of the nine lifestyle areas of food, drink, movement, mind, wear, space, rest, self-care and social collaboration is also generated, activated and controlled by the system. A communication module enables instant messaging and email servers to send text, audio reminders and prompts automatically when deviation from the individual wellness protocol is recognized by the system.

A system and method are disclosed of exemplary embodiments for providing navigation and control of lifestyle/wellness protocols generated by expert systems. The present invention operates on smart phones, tablet computers and desktop systems. By interacting with the icons for: food, drink, movement, mind, wear, space, rest, self-care, and social collaboration, personalized lifestyle/wellness protocols, products and services, and communications are engaged by the system.

When wellness protocols are received by users the system communicates via audio, text, and graphics to monitor protocol compliance on digital devices, smartphone, tablets or desktops.

The system determines system adherence to wellness protocols and ensures compliance is maintained. On a real-time basis the system monitors lifestyle activity and adjusts individual protocols goals. Interactions are monitored and data compiled over time. Deviation from the individualized wellness protocol automatically triggers communication from the system to enable and correct adherence to protocols.

For various types of actions the system can generate qualitative and quantitative metrics such as whether or not a protocol task was completed; the start and end time of the task; and, if the task was repeated and when. Information about the frequency of interaction with the system is also captured, as well as, the effectiveness of the messages that are sent to ensure adherence. Data related to time on the system, the navigation in the system and the operations performed are also captured.

In regard to backend databases, the system includes the following:

1) A wellness protocol generation engine (relational database) wherein the input of wellness and lifestyle data and information are mapped against optimal lifestyle wellness databases in the system to determine needs in the wellness/lifestyle areas of food, drink, movement, mind, wear, space, rest, self-care and social collaboration;

2) Directories of products and services for purchase. Data regarding system interest in wellness products and services is stored along with data on products and services ordered and used in the wellness/lifestyle areas of food and nutrition, drink, movement, mind, wear, space, rest, self-care and social collaboration;

3) Protocol instructional content in each of the nine lifestyle areas of food, drink, movement, mind, wear, space, rest, self-care and social collaboration.

4) A communication database module that sends text, audio reminders and prompts to systems automatically when deviation from the individual wellness protocol is recognized. The communication module also connects with wellness providers and third parties as necessary to provide data to wellness practitioners and enterprises for clinical, research and diagnostic purposes.

A preferred embodiment the methodology comprises digital (online) wellness lifestyle questionnaires and biological test data and genetic data to construct wellness protocols by the system (See Table 1 below). The wellness/lifestyle questionnaires are designed to reveal a large volume of information including biological test data and genetic data related to personal wellness characteristics. To build each individualized wellness protocol the system provides the means to enter data regarding usual lifestyle choices and other biological and genetic relevant information. The personalized wellness protocols are based on consideration of existing food, drink, movement, mind, wear, space, rest, self-care and social collaboration lifestyle data and information and their influence on epigenetic change. From this data and information the system identifies the dissimilarity between current lifestyles and the database of optimal wellness lifestyles in each of nine lifestyle areas. As a result of this process individualized wellness protocols are created by the system. Once created, accordingly, individualized wellness protocols are tailored by the system and electronically delivered with goals and a timeframe for the protocol execution and completion.

Once created, accordingly, individualized wellness protocols are personalized by the system and electronically delivered with goals and a timeframe for the protocol.

The system determines system adherence to wellness protocols and communicates to ensure compliance is maintained. On a real-time basis the system monitors lifestyle activity and adjusts individual protocols to help users reach personal goals. System interactions are also monitored and data compiled according to the users reported involvement with the wellness protocol over time. Deviation from the individualized wellness protocol automatically triggers communication from the system to the user.

In a preferred embodiment of the present invention, it is manifested as an application that can be downloaded to a desktop computer, computer tablet, or a smart phone consisting of a system interface associated with lifestyle/wellness protocols and allowing input from lifestyle/wellness questionnaires and health assessment information including biological test data and genetic data. The systems interface facilitates system integration and interaction options allowing input, access, to databases and protocols, and communications.

In another embodiment of the present invention, it may also be used independently of the expert systems component to store, access, review and modify data external to the system that is relevant to lifestyle/wellness protocols of interest to the user.

Exemplary embodiments enable creation, control and navigation of lifestyle/wellness protocols generated by systems comprising of: a wellness protocol generation engine (relational database) wherein the input of wellness information identifying a user and a description of a user's lifestyle and biometrics is mapped against optimal lifestyle wellness databases in the system to determine the user's wellness needs in the lifestyle areas of food, drink, movement, mind, wear, space, rest, self-care and social collaboration; and against directories of products and services for systems to purchase lifestyle and wellness products and service. Data file directories regarding user interest in wellness products and services are stored along with data on products and services ordered and used in the lifestyle areas of food and nutrition, drink, movement, mind, wear, space, rest, self-care and social collaboration. Protocol instructional content in each of the nine lifestyle areas of food, drink, movement, mind, wear, space, rest, self-care and social collaboration is also generated by the system and controlled by the user. A communication module enables instant messaging and email servers send text, audio reminders and prompts automatically when deviation from the individual wellness protocol is recognized by the system.

The terms and expressions which have been used to describe the disclosure herein are used merely as terms of description and not as limitations in recognition of the fact that various modifications are possible within the scope of the invention claimed. That is, the invention is not intended to be limited to the embodiments disclosed recognizing the disclosure is capable of being further expressed through additional and future embodiments that employ this teaching.

Having described our invention, we claim:

1-13. (canceled)

14. A systems interface component for navigating and controlling wellness expert systems applications, services or computing devices, activating wellness protocols generated by expert systems algorithmic analysis of user quantitative and qualitative input data parameters in nine lifestyle areas: food, drink, movement, mind, wear, space, rest, self-care and social collaboration, comprising:
a. A mechanical device layer including a plurality of touch sensitive and device sensitive input controls actionable by a user to provide a corresponding set of input functionality for controlling the wellness application, service or computing device, wherein each mechanical input control maps to an independent wellness function in nine lifestyle areas: food, drink, movement, mind, wear space, rest, self-care an social collaboration. and;
b. Wherein the plurality of mechanical input controls includes nine mechanical input controls arranged in a circular clockwise array and mapping to icons starting with food, followed clockwise by drink, move, mind, wear, space, sleep, self-care and ending with social collaboration, before completing the array and returning to food. Each of which enable access to protocols, products and services.
c. Wherein the plurality of mechanical input controls includes a centrally located mechanical input control mapping to a central icon enabling access to external databases and Application Program Interfaces (APIs) to disparate systems.

15. A client computing device (smart phone, laptop computer, tablet computer, desktop or other computing device) including the systems interface component of claim 14.

16. A method for receiving user input including, but not limited to, wellness and health history questionnaires and bio-medical data from a user or health professional via the systems interface component including a touch or device sensitive input layer including a plurality of touch or device sensitive data input controls, controlling wellness expert systems applications, services or computing devices and activating wellness protocols generated by expert systems algorithmic analysis of user input data parameters in nine lifestyle areas: food, drink, movement, mind, wear, space, rest, self-care and social collaboration.

17. A method for accessing and integrating wellness protocol content and interactions with the system interface comprising the steps of:
a. Selecting a protocol for establishing a navigation session between said systems interface and a protocol content online and offline;
b. Transmitting data from protocol databases to an on-line service server or off-line storage system from a server or device in communication with said interface;
c. Retrieving content associated with said wellness protocol, on-line service server adapted to retrieve said content;
d. Creating a display page containing said retrieved wellness protocol content; and,
e. Presenting said display page in a window independent from other windows displayed by said interface, said presentation displayed by said systems interface.

18. The method of claim 17 further comprising the steps of:
a. Locating a plurality of members of a social collaboration wellness community associated with said wellness expert systems applications, services or computing devices, said plurality of members located by said on-line service server; and,
b. Initiating and managing interactions with one or more of said plurality of members of said community associated with social collaboration wellness community associated with said wellness expert systems applications, services or computing devices, said interactions initiated and managed by said systems interface and said communications server;
c. The method of claim 17 wherein said systems interface, said client computing device, and said communications server communicate via an Internet protocol, instant messaging and email

19. A method of claim 17 activating wellness protocols generated by algorithmic analysis of user quantitative and qualitative input parameters in nine lifestyle areas: food, drink, movement, mind, wear, space, rest, self-care and social collaboration using a graphical systems interface and input device to navigate internal and external databases and content, said method comprising:
a. A computer readable medium having program code recorded thereon for execution on a device for displaying wellness protocols relating to and facilitating modification of lifestyle in nine wellness areas of: food, drink, movement, mind, wear, space, rest, self-care and social collaboration;
b. The program code causing a computer device to take as input lifestyle/wellness user data stored in an electronic system and to generate as output recommendations for therapeutic interventions that are defined by applicable protocols for food, drink, movement, mind, wear, space, rest, self-care and social collaboration on a graphical system interface;

20. A method of claim 17 for activating wellness protocols generated by expert systems algorithmic analysis of user qualitative and quantitative input data parameters in nine lifestyle areas: food, drink, movement, mind, wear, space, rest, self-care and social collaboration using a graphical systems interface and input device (mechanical pointing or touch) for facilitating displaying and navigating lifestyle/wellness protocols and ordering related products, services and conducting social collaboration;
